(19) United States
(12) Patent Application Publication
(10) Pub. No.: US 2025/0349389 A1
(43) Pub. Date: Nov. 13, 2025

(54) NUCLEIC ACID LIBRARY SEQUENCING TECHNIQUES WITH ADAPTER DIMER DETECTION

(71) Applicant: Illumina Cambridge Limited, Cambridge (GB)

(72) Inventors: Carla Botas-Sanmartin, London (GB); Isabelle Marie Julia Rasolonjatovo, Haverhill (GB); Andrea Sabot, Cambridge (GB)

(21) Appl. No.: 18/553,710

(22) PCT Filed: Mar. 31, 2022
(86) PCT No.: PCT/EP2022/058598

§ 371 Date: Oct. 2, 2023

Related U.S. Application Data
(60) Provisional application No. 63/168,762, filed on Mar. 31, 2021.

(51) Int. Cl. G16B 35/20; C12Q 1/6855; C12Q 1/6869; C12Q 1/6876; G16B 20/10

(52) U.S. Cl. CPC G16B 35/20; C12Q 1/6855; C12Q 1/6869; C12Q 1/6876; G16B 20/10

(57) ABSTRACT

A library sequencing technique with library quality control metrics is described. Sequence data using a sequencing primer that is complementary to a common adapter sequence in fragments of a nucleic acid sequencing library. The sequencing primer excludes a 3′ terminal nucleotide of the common adapter sequence at a junction with a fragment insert. This exclusion avoids a mismatch region in any adapter dimers present in the sequencing library, and the sequence data includes adapter dimer sequence data, which is used to generate the quality control metrics.

### CROSS-REFERENCE TO RELATED APPLICATIONS

The present application is a national stage application claiming priority to PCT/EP2022/058598, entitled “NUCLEIC ACID LIBRARY SEQUENCING TECHNIQUES WITH ADAPTER DIMER DETECTION” and filed on Mar. 31, 2022, which claims priority to and the benefit of U.S. Provisional Application No. 63/168,762, entitled “NUCLEIC ACID LIBRARY SEQUENCING TECHNIQUES WITH ADAPTER DIMER DETECTION” and filed on Mar. 31, 2021, the disclosure of which is incorporated by reference in its entirety herein for all purposes.

### REFERENCE TO SEQUENCE LISTING

This application includes an electronically submitted sequence listing in.txt format. The.txt file contains a sequence listing entitled “IP-2139-US_ST25.txt” created on Apr. 22, 2024 and is 7,143 bytes in size. The sequence listing contained in this.txt file is part of the specification and is hereby incorporated by reference herein in its entirety.

### BACKGROUND

The technology disclosed relates generally to nucleic acid sequencing techniques. In particular, the technology disclosed relates to sequencing workflows for nucleic acid sequencing that include a detection and/or characterization of adapter dimers formed during library preparation.

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Sample preparation (e.g., library preparation) for next-generation sequencing can involve fragmentation of nucleic acids, such as genomic DNA or double-stranded cDNA (prepared from RNA) into smaller fragments, followed by addition of functional adapter sequences to the strands of the fragments. Such adapters may include priming sites for DNA polymerases for sequencing reactions, restriction sites, and domains for capture, amplification, detection, address, and transcription promoters. In certain techniques, the adapter are added to ends of the nucleic acid fragments by ligation to yield fragments with adapters at both ends.

One drawback in preparing nucleic acid fragment libraries by ligating adapters to the ends of template nucleic acid fragments is the formation of adapter dimers. Adapter dimers are undesirable side products formed by the ligation of two adapters directly to each other such that they do not contain an intervening template nucleic acid fragment as an insert. In some sequencing techniques, adapter dimers present in the nucleic acid fragment library are amplified when the library is amplified, e.g., as part of a sequencing workflow. Since adapter dimers are generally smaller than the fragments contained in the libraries, they can amplify and accumulate at a faster rate, thus contaminating the sequencing results with adapter dimer reads that are not representative of the sample. In other techniques, the adapter dimers are not amplified and/or sequenced, because the adapter dimers are formed with a mismatch between the adapter dimer and the sequencing primers that are complementary to the adapters. Certain sequencing polymerases will not tolerate the mismatch and, therefore, will not amplify or sequence the adapter dimers. However, even when the adapter dimers are not sequenced, the presence of adapter dimers in the library may result in lower quality sequencing results. In the case of clustered arrays, a lower density of meaningful insert sequence data is obtained from a chip of finite size if a significant population of clusters are occupied by adapter dimers and, therefore, have no sample DNA sequence. Thus, the preparation of libraries with a low level of adapter-dimers is advantageous in the sequencing of polynucleotides, particularly when such processes are high-throughput. Described herein are techniques for assessing adapter dimers present in a nucleic acid fragment library to facilitate improvement of nucleic acid sequencing from such libraries.

### BRIEF DESCRIPTION

In one embodiment, the present disclosure relates to a method of characterizing a nucleic acid library that includes the steps of sequencing a nucleic acid library using a sequencing primer to generate sample sequencing data representative of fragments of the nucleic acid library and of adapter dimer sequencing data, wherein an individual fragment of the nucleic acid library comprises a sample insert flanked by first adapters; wherein an individual adapter dimer of the nucleic acid library comprises second adapters ligated directly to each other at a junction, wherein the first adapters and the second adapters have a same sequence, wherein the sequencing primer is identical to a portion of the same sequence and wherein the individual adapter dimer comprises a mismatch region at the junction and wherein the sequencing primer, when bound to a strand of the individual adapter dimer, has a 3′ terminus that is 5′ of the junction; and determining a quality metric of the nucleic acid library based on the adapter dimer sequencing data.

In another embodiment, the present disclosure relates to a method of characterizing a nucleic acid library that includes the steps of receiving, at a sequencing device, an input that a sequencing run of a pool of a plurality of nucleic acid libraries is an adapter dimer quality control sequencing run; causing the sequencing device to generate sequence data from the pool using a sequencing primer that is complementary to a common adapter sequence in fragments of the plurality of nucleic acid libraries and that excludes a 3′ terminal nucleotide of the common adapter sequence at a junction with a fragment insert; calculating quality metrics for each individual nucleic acid library, wherein the quality metrics comprise a percentage of adapter dimers in each individual nucleic acid library; and identifying a subset of nucleic acid libraries of the plurality of nucleic acid libraries with a percentage of adapter dimers above a specification limit.

In another embodiment, the present disclosure relates to a sequencing device that includes a flow cell having loaded thereon a pool of a plurality of nucleic acid libraries and a sequencing primer that is complementary to a common adapter sequence in fragments of the plurality of nucleic acid libraries and that excludes a 3′ terminal nucleotide of the common adapter sequence at a junction with a fragment insert. The sequencing device also includes a computer programmed to receive an input that a sequencing run of the pool is an adapter dimer quality control sequencing run; cause the sequencing device to generate sequence data from the pool using the sequencing primer; calculate quality metrics for each individual nucleic acid library to determine a percentage of adapter dimers in each individual nucleic acid library; and identify a subset of nucleic acid libraries of the plurality of nucleic acid libraries with a percentage of adapter dimers above a specification limit

The preceding description is presented to enable the making and use of the technology disclosed. Various modifications to the disclosed implementations will be apparent, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein. The scope of the technology disclosed is defined by the appended claims.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic illustration of a method for preparing a nucleic acid library, in accordance with aspects of the present disclosure;

FIG. 2 is a schematic illustration of a method for generating sequencing reads from a nucleic acid library, in accordance with aspects of the present disclosure;

FIG. 3 is a schematic illustration of sequencing primer location relative to the fragment adapter and insert;

FIG. 4 is a schematic illustration of a method for preparing a nucleic acid library, in accordance with aspects of the present disclosure;

FIG. 5 is a schematic illustration of a method generating sequencing reads from a nucleic acid library, in accordance with aspects of the present disclosure;

FIG. 6 is a schematic illustration of a nucleic acid sequencing workflow, in accordance with aspects of the present disclosure;

FIG. 7 shows sequencing results for rebalanced nucleic acid libraries, in accordance with aspects of the present disclosure;

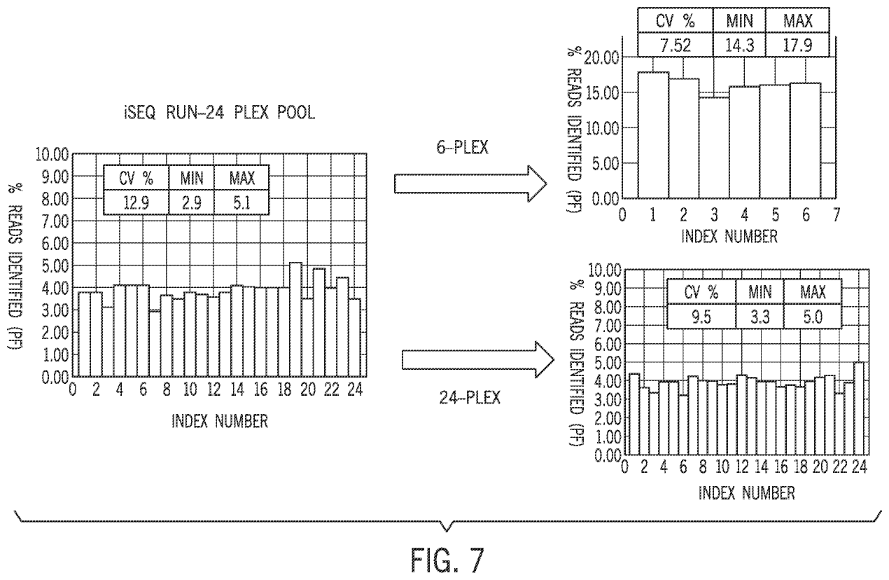

FIG. 8 shows sequencing results for rebalanced nucleic acid libraries, in accordance with aspects of the present disclosure;

FIG. 9 shows example comparisons between quality metrics using sequenced adapter dimers and PCR results for the same sample, in accordance with aspects of the present disclosure; and

FIG. 10 is a block diagram of a sequencing device configured to acquire sequencing data in accordance with the present techniques.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

Library preparation for downstream processing and analysis, such as for nucleic acid sequencing, generally involves fragmenting a nucleic acid (e.g. genomic DNA) to generate fragments (e.g., nucleic acid fragments) that are subsequently amplified and sequenced. Relying on quantification techniques alone, such as quantitative PCR (Q-PCR), to measure the template yield of the library preparation does not give information on the quality of the library and does not provide standardized quality metrics that estimate presence of the correct insert size, sequencing and clustering performance of the library, and/or presence of contaminants or overrepresented sequences such as adapter dimers.

A quality control using sequencing is a powerful approach to identify any potential issues with a library. Provided herein is a sequencing workflow that generates library quality metrics based on sequencing data that is representative of library fragments as well as adapter dimers. In an embodiment, the quality metrics may include one or more of sequencing performance (e.g., Q30 scores), % adapter dimers, insert size, yield per sample (DNA concentration), % duplicates, number of aligned reads and clustering performance (% cluster pass filter and % occupancy). The disclosed techniques provide improvements over other techniques that identify adapter insert size and a percentage of adapter dimers by looking at the presence of off-size elements in the library, but that do not use adapter dimer sequence data.

The disclosed techniques use sequencing primers that are selected by a design-guided approach and that generate sequencing data representative of the adapter dimers present in a particular sequencing library preparation. This adapter dimer sequence data is identified and provided as input to quality metrics for an individual sequencing library. In an embodiment, the quality metrics may in turn be used to guide library normalization or rebalancing steps. The disclosed techniques are in contrast to sequencing workflows that use sequencing primers that, when hybridized to an adapter dimer, have a mismatch between the 3′ terminal nucleotide of the primer and the adapter dimer caused by sequence differences between insert-containing fragments and adapter dimers. When using polymerases having low tolerances for mismatches, e.g., stringent or mismatch-intolerant polymerases, the mismatch prevents the adapter dimers from being sequenced. Therefore, the acquired sequencing data from a library that includes adapter dimers does not include any adapter dimer sequencing reads that can be characterized as provided herein. However, even if the adapter dimers are not represented in such sequencing data, their presence nonetheless may be associated with poor library quality metrics. Further, the use of mismatch-intolerant polymerase is desirable to generate accurate sequencing results from the sample nucleic acid. Accordingly, the disclosed techniques permit characterization of adapter dimers in a sequencing library based on sequencing data and also generate such data using mismatch-intolerant polymerases.

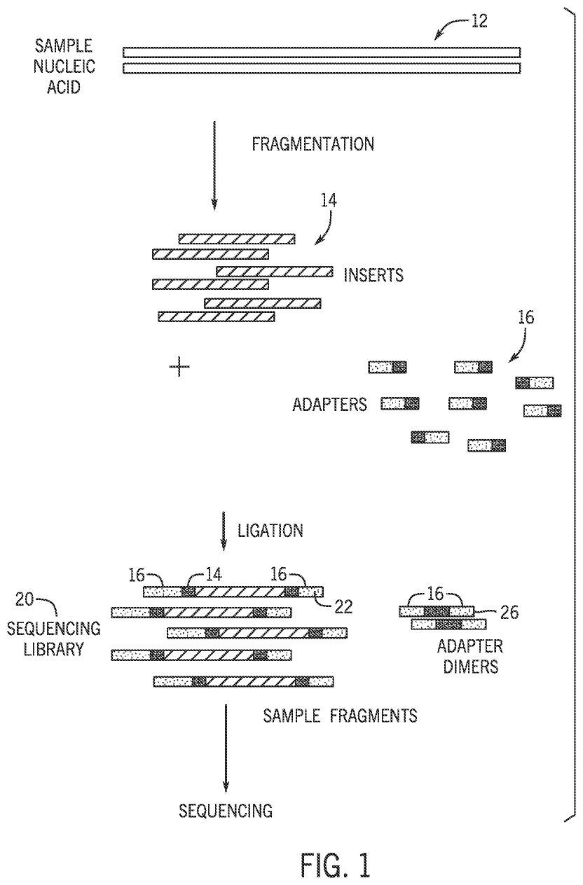

FIG. 1 is a schematic illustration of a library preparation technique from sample nucleic acid 12. The sample nucleic acid 12 is fragmented to generate nucleic acid inserts 14 according to suitable fragmentation techniques, such as sonication, enzyme treatment, etc. The generated inserts 14 are ligated to adapters 16, as generally disclosed herein, to generate a sequencing library 20 that includes adapter end-ligated fragments 22 that generally have an adapter-insert-adapter arrangement. That is, the inserts 14 are flanked by adapters 16. The fragments 22 of the sequencing library 20 may share common sequences at their 5′ ends and common sequences at their 3′ ends. That is, the common sequences are from common adapters 16, which may be all of a same type or of a same sequence, and may be ligated to ends of the inserts 14 in the appropriate orientation.

In addition, the sequencing library 20 may include adapter dimers 26, which are adapters 16 that are ligated to one another directly and that do not include an intervening insert 14. The adapter dimers 26 are contaminants or undesired elements of the sequencing library 20.

Once prepared, the sequencing library 20 is provided to a sequencing platform to generate sequencing data from adapter dimers present in the sequencing library 20 that can be used to improve sequencing results or drive cleanup, rebalancing, or other enrichment steps that may be used to generate improved sequencing data of the sample nucleic acid 12. The quality of an individual sequencing library 20 may be related to the quality of the starting sample nucleic acid 12, the concentration of the sample nucleic acid 12, operator variability in performing library preparation workflow steps, reagent quality, adapter concentration, etc. Therefore, different libraries 20 may have different qualities relative to one another. The disclosed techniques generate quality metrics specific for respective individual libraries 20.

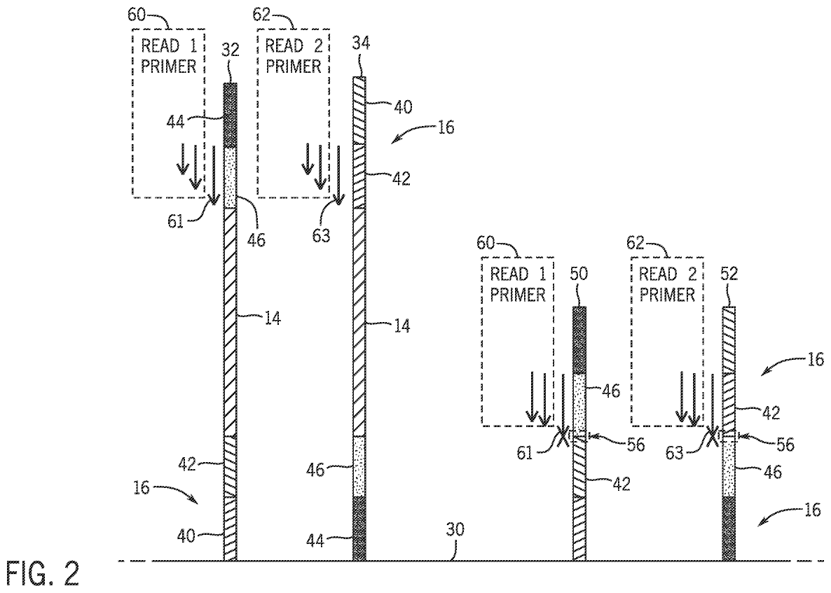

FIG. 2 is a schematic illustration of a paired end sequencing that may be performed with the sequencing library 20 and using the sequencing primers that generate the adapter dimer sequencing information. It should be understood that the disclosed techniques may additionally or alternatively be used with single-end sequencing runs. Further, while FIG. 2 illustrates sequencing primers for forward and reverse strands being present simultaneously, it should be understood that paired end sequence steps are performed in series to generate sequencing data, and that additional sequencing steps to sequence indexes may also be performed in series.

The sequencing may be performed on a substrate 30, such as a chip, flow cell, or solid substrate. In other embodiments, the sequencing may be performed on a bead. The substrate 30 includes immobilized forward strands 32 and reverse strands 34 of the sample fragments 22. The strands 32, 34 may be part of clusters formed by bridge amplification such that each cluster or site on the substrate 30 is representative of a single insert 14 derived from the sample 12. Different sites associated with different locations on the substrate have different captured sample fragments 22 with different inserts 14. Both strands 32, 34 are flanked by adapter sequences. As illustrated, the adapter sequences are single-stranded versions of the adapter 16 such that the 5′ adapter of the forward strand is located 3′ of the adapter on the reverse strand and vice versa. Thus, the 5′ sequence and the 3′ sequence on each strand may be distinguishable. The adapter sequences may include a capture region 40, 44 that permits capture by immobilized capture oligonucleotides on the substrate 30. The adapter sequences also include a primer region 42, 46.

A forward strand 50 and a reverse strand 52 from the adapter dimers 26 are also captured on the substrate 30 via the capture regions 40, 44. The primer regions 40, 44 are directly ligated to one another. The insert-containing forward strand 32 and the adapter dimer forward strand 50 are sequenced as part of a sequencing workflow by extension from a sequencing primer that is complementary to and binds to the primer region 46. As illustrated, the read 1 primer 60 is designed to avoid a mismatch region 56 that is located at the junction or dimerization location of the adapter dimer 26. That is, the mismatch region 56 is or includes a location where a first adapter 16 and a second adapter 16 join to one another. The read 1 primer 60 has a 3′ terminus that is located 5′ of the mismatch region 56. In an embodiment, the mismatch region 56 is a single nucleotide, is 2-3 nucleotides, or 2-10 nucleotides. The mismatch region is generated because the dimerization process results in a different sequence in the adapter dimer 26 relative to the sample fragment 22 that is reflected in strands generated from the library 20. There is no mismatch region 56 in the strands 32, 34 because the insert 14 is ligated at respective ends of the adapters 16.

The design-guided sequencing primers that generate the adapter dimer sequencing information include a read 1 primer 60. Because the conventional primer 61 includes the mismatch region 56, the conventional primer is not capable of extending, and generating sequencing data, from the adapter strand 50. Accordingly, the read 1 primer 60 is at least distinguishable from the conventional sequencing primer based on a different 3′ nucleotide. In an embodiment, the read 1 primer 60 is a truncated version of the conventional primer 61 that does not include the last 3′ nucleotide but that includes all other nucleotides. In an embodiment, the read 1 primer 60 is a shifted version of the conventional primer 61 (FIG. 2) that does not include the last 3′ nucleotide.

The read 1 primer 60 can be a single primer sequence selected from a set of potential primers, as illustrated, that avoid the mismatch region 56. In an embodiment, the read 1 primer 60 is designed to have a 3′ end that, when hybridized to the forward strand 32, extends from a location close to the insert 14, e.g., within 10 nucleotides of the insert 14. In an embodiment, the read 1 primer 60 extends from a location within three nucleotides of the insert 14. Additionally or alternatively, the read 1 primer 60 may be designed to avoid or not include other functional regions of the adapter 16, such as an index region, a barcode region, and/or a capture region 44. The read 1 primer 60 may be between 18 and 24 nucleotides in length. In an embodiment, the read 1 primer 60 complementary to the primer region 46 for the forward strand 32 is at least 50%, at least 75%, or at least 95% identical to the sequence of primer region 42 on the reverse strand 34.

In the paired-end embodiment, the sequencing primers also include a read 2 primer 62. Because the conventional primer 63 includes the mismatch region 56, the conventional primer is not capable of extending, and generating sequencing data, from the adapter strand 52. Accordingly, the read 2 primer 62 is at least distinguishable from the conventional sequencing primer based on a different 3′ nucleotide. The read 2 primer 62 has a 3′ terminus that is located 5′ of the mismatch region 56. In an embodiment, the read 2 primer 62 is a truncated version of the conventional primer 63 that does not include the last 3′ nucleotide but that includes all other nucleotides. In an embodiment, the read 2 primer 62 is a shifted version of the conventional primer 63 that does not include the last 3′ nucleotide and that is shifted one nucleotide in the 5′ direction. The read 2 primer 62 can be a single primer sequence selected from a set of potential primers, as illustrated, that avoid the mismatch region 56. In an embodiment, the read 2 primer 62 is designed to have a 3′ end that, when hybridized to the reverse strand 34, extends from a location close to the insert 14, e.g., within 10 nucleotides of the insert 14. In an embodiment, the read 2 primer 62 extends from a location within three nucleotides of the insert 14. Additionally or alternatively, the read 2 primer 62 may be designed to avoid or not include other functional regions of the adapter 16, such as an index region, a barcode region, and/or a capture region 40. The read 2 primer 62 may be between 18 and 24 nucleotides in length. In an embodiment, the read 2 primer 62 complementary to the primer region 42 for the reverse strand 34 is at least 50%, at least 75%, or at least 95% identical to the sequence of primer region 46 on the forward strand 32.

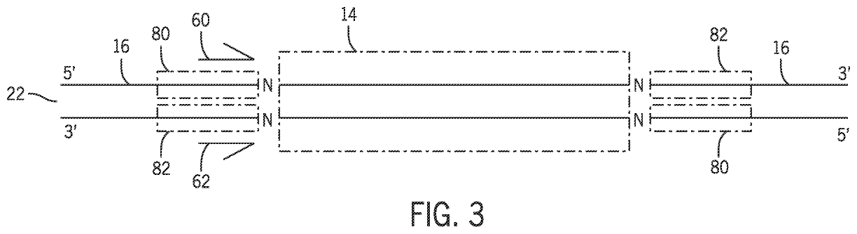

FIG. 3 is a schematic illustration of a position of the read 1 primer 60 and the read 2 primer 62 in the adapter 16 and relative to a position of the insert 14. The primer 60 corresponds to the region 80 on the fragment 22 illustrated as N in FIG. 3, corresponding to the nucleotide at the interface between the insert 14 and the adapter 16. In an embodiment, provided are adapter-dimer capable sequencing primers that have a sequence as follows:

### Read 1 Primer 60:

A sequence including 15-25 nucleotides in the primer region 80 and 5′ but not including the terminal 3′ nucleotide N of the adapter 16. In an embodiment, the terminal nucleotide Nis a “T”.

### Read 2 Primer 62:

A sequence including 15-20 nucleotides in the primer region 82 and not including the nucleotide 3′ of the insert 14. In an embodiment, the terminal nucleotide N is an “A”.

The read 1 primer 60 and the read 2 primer 62 are close to but, in an embodiment, one nucleotide separated from the insert 14 such that the sequence information generated within the insert 14 is maximized.

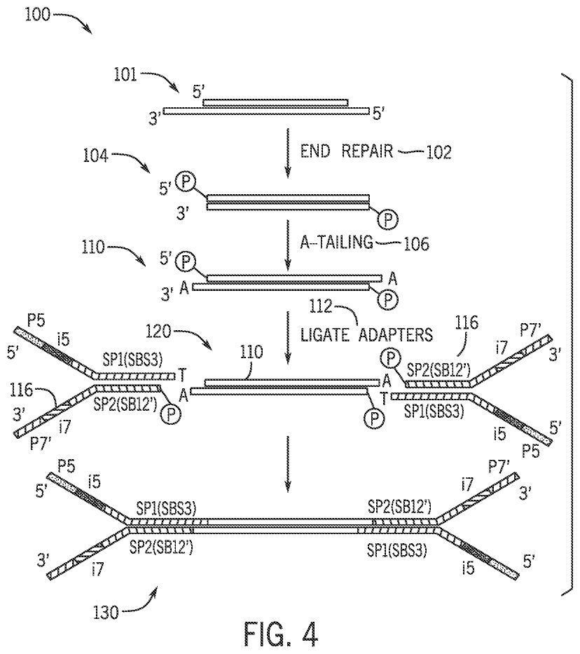

FIG. 4 shows an example library preparation workflow 100 using forked adapters and that may be used in conjunction with the disclosed techniques. Although only one double-stranded fragment 101 is illustrated, thousands to millions of fragments of a sample can be prepared simultaneously in the workflow. DNA fragmentation by physical methods produces heterogeneous ends, comprising a mixture of 3′ overhangs, 5′ overhangs, and blunt ends. The overhangs will be of varying lengths and ends may or may not be phosphorylated. An example of the double-stranded DNA fragments obtained from fragmenting genomic DNA of operation is shown as fragment 101. Fragment 101 has both a 3′ overhang on the left end and a 5′ overhang shown on the right end. If DNA fragments are produced by physical methods, the workflow proceeds to perform end repair operation 102, which produces blunt-end fragments having 5′-phosphorylated ends. In some implementations, this step converts the overhangs resulting from fragmentation into blunt ends using T4 DNA polymerase and Klenow enzyme. The 3′ to 5′ exonuclease activity of these enzymes removes 3′ overhangs and the 5′ to 3′ polymerase activity fills in the 5′ overhangs. In addition, T4 polynucleotide kinase in this reaction phosphorylates the 5′ ends of the DNA fragments. The fragment 104 is an example of an end-repaired, blunt-end product.

After end repairing, workflow 100 proceeds to adenylating 3′ ends of the fragments (step 106), which is also referred to as A-tailing or dA-tailing, because a single dATP is added to the 3′ ends of the blunt fragments to prevent them from ligating to one another during the adapter ligation reaction. Double stranded molecule 110 shows an A-tailed fragment having blunt ends with 3′-dA overhangs and 5′-phosphate ends. A single ‘T’ nucleotide on the 3′ end of each of the two sequencing adapters 116 provides an overhang complementary to the 3′-dA overhang on each end of the insert for ligating the two adapters to the insert. In an embodiment, the read 1 primer 60 and the read 2 primer exclude the single “T” nucleotide.

After adenylating 3′ ends, workflow 100 proceeds to ligating (step 112) oligonucleotides, e.g., adapters 116, to both ends of the fragments 110. The adapters 116 may include index sequences for identifying individual samples in a multiplexed reaction. The P5 and P7′ oligonucleotides are common or universal adapters in all of the samples of a multiplexed reaction and are complementary to the amplification primers bound to the surface of flow cells of the Illumina sequencing platform, and are also referred to as amplification primer binding site. They allow the adapter-insert-adapter library to undergo bridge amplification. Other designs of adapters and sequencing platforms may be used in various implementations. The adapters 116 also include two sequence primer binding sequences for Read1 and Read2. Other sequencing primer binding sequences may be included in the adapters for different reactions, e.g., index reads.

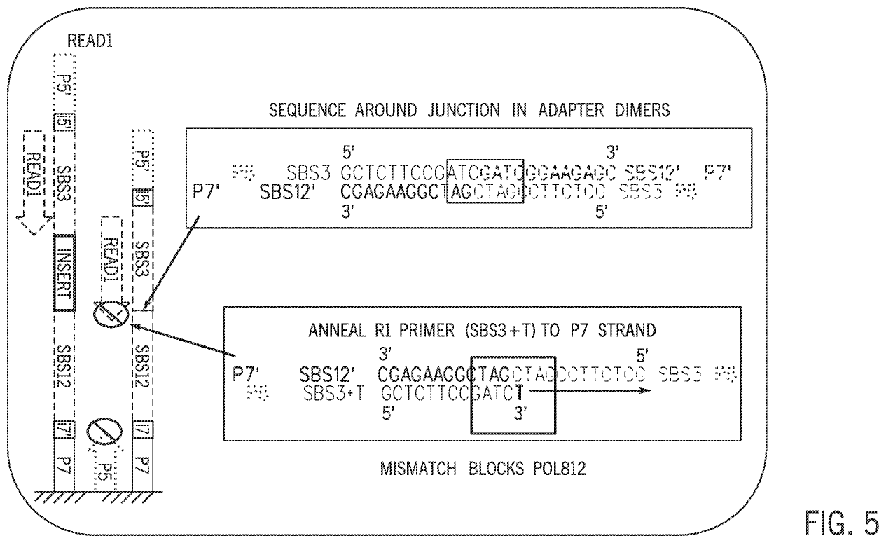

In an embodiment, the disclosed techniques may be used to detect adapter dimers using iSeq100 in Truseq PCR-FREE library preparations (Illumina, Inc.). The custom recipe and primers are used in this protocol to enable this adapter dimer detection on iSeq (Illumina, Inc.). iSeq DNA sequencing polymerase pol812 (SED ID NO: 1), which cannot sequence the adapter dimers when there is a mismatch (T-C) between the last nucleotide (T) of the read primers and the first readable nucleotide of the adapter dimer (C), as shown in FIG. 5. That is, the read 1 primer in FIG. 4 is not included in the set of contemplated read 1 primers 60 (FIG. 2), but is a conventional primer 61. Accordingly, provided herein is a custom read 1 primer without the “T” at the end of SBS3 (read 1 primer). Also provided herein is a SBS12 (read 2 primer) without the “T” at the end. These primers can be used to detect adapter dimers. Although the adapters and the sequencing process described here are based on the Illumina platform, other adapters and sequencing technologies may be used instead of or in addition to the Illumina platform.

The disclosed techniques may be used to qualify, rebalance, normalize and quantify libraries using certain sequencing platforms, such as the iSeq platform, the NextSeq platform, and/or the NovaSeq (Illumina, Inc.) that use a mismatch-intolerant polymerase. As provided herein, an example of a mismatch-intolerant polymerase is disclosed at SEQ ID NO:1, and is also referred to herein as the Pol812 polymerase. Other mismatch intolerant or high fidelity polymerases that may be used in conjunction with the disclosed techniques include pfu polymerase or Q5 polymerase. However, it should be understood that other sequencing polymerases may be used in conjunction with the disclosed techniques, including relatively mismatch-tolerant sequencing polymerases. That is, because the discloses techniques provide primers that avoid adapter dimer mismatches, a wider variety of sequencing polymerases are able to generate adapter dimer sequencing data as provided herein.

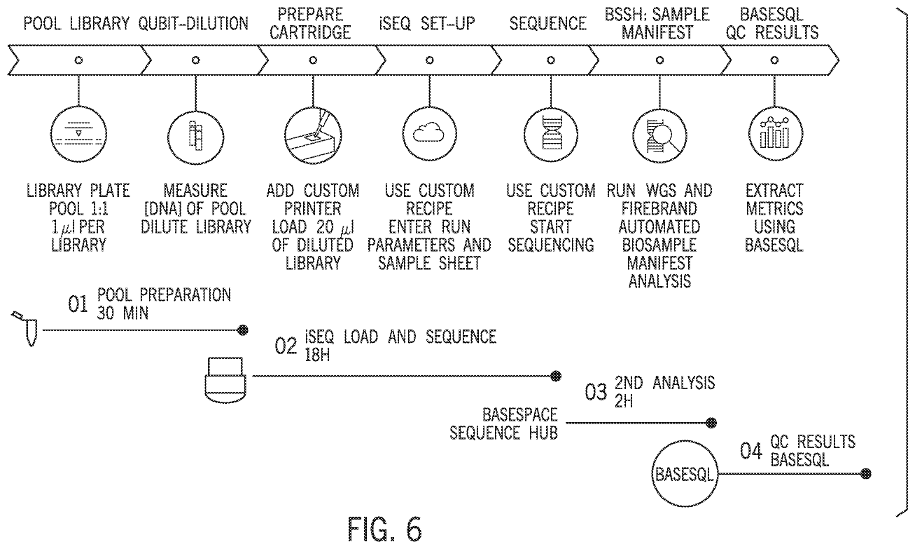

FIG. 6 is an example sequencing workflow for the iSeq platform according to the disclosed embodiments that automatically generates quality metrics for a sequencing library. The workflow initiates after the library preparation workflow (e.g., as shown in FIG. 1 and FIG. 4). The prepared libraries can be pooled at a 1:1, with a recommended volume of 1 μl per sample. Dilution can be performed based on a measurement of DNA concentration, such as the Illumina Qubit technique, and the library pool is to the appropriate concentration based on the DNA concentration. However, in an embodiment, DNA concentration estimates or other quality metrics generated from adapter dimer sequencing data may replace direct DNA measurement, such as measurement via Qubit. This provides the benefit of speeding up the workflow by eliminating a time-consuming DNA measurement step. Further, acquiring the adapter dimer sequencing data occurs during the sequencing of the library, such that the disclosed quality metrics do not add time to the workflow and may reduce the overall time of the workflow. Accordingly, the disclosed techniques permit more efficient operation of the sequencing device.

The custom primer sequences for the read 1 primer 60 and the read 2 primer 62 can be the following:

SBS3 Read 1
(SEQ ID NO: 2)
ACACTCTTTCCCTACACGACGCTCTTCCGA
SBS12 Read 2
(SEQ ID NO: 3)
GTGACTGGAGTTCAGACGTGTGCTCTTCCGATC
SBS3 Read 1
(SEQ ID NO: 4)
ACACTCTTTCCCTACACGACGCTCTTCCG
SBS12 Read 2
(SEQ ID NO: 5)
GTGACTGGAGTTCAGACGTGTGCTCTTCCGAT
SBS3 Read 1
(SEQ ID NO: 6)
ACACTCTTTCCCTACACGACGCTCTTCC
SBS12 Read 2
(SEQ ID NO: 7)
GTGACTGGAGTTCAGACGTGTGCTCTTCCGA

The adapter dimer-capable sequencing primers, such as primers including the sequences SEQ ID NO:2 and SEQ ID NO:3, SEQ ID NO:4 and SEQ ID NO:5, SEQ ID NO:6 and SEQ ID NO:7, or other combinations of these sequences that include a read 1 primer and a read 2 primer, can be added to the sequencing substrate, e.g., the flow cell. When these primers are used, the sequencing device can be programmed to operate according to an adapter dimer metrics mode based on an input indicating that the adapter dimer-capable sequencing primers are in use. When conventional primers are used, a different operating mode that does not provide these metric is selected. It should be understood that these primer sequences are by way of example, and other primers based on other adapter sequences may also be used. In other examples, the primer sequences are based on read 1 and read 2 sequencing primer pairs for other Illumina technologies, or other NGS sequencing technologies.

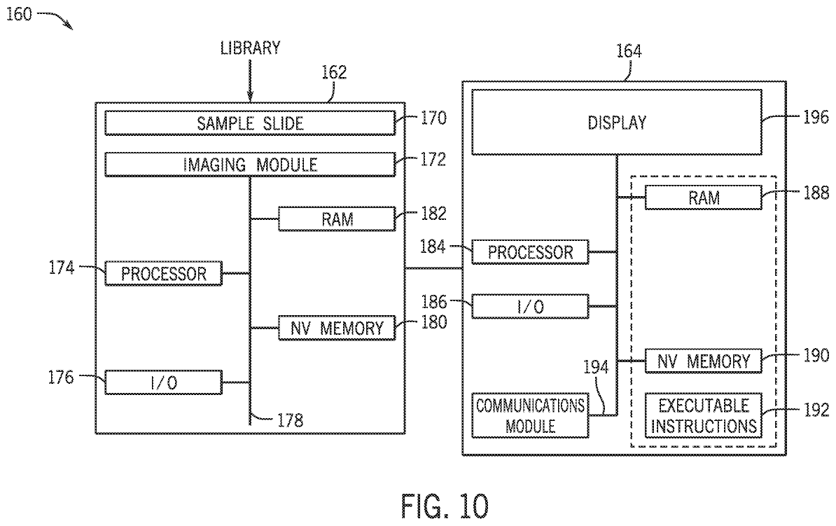

Once the sequencing run is finished running, it will automatically generate one or more quality metrics reports that are provided to a computer (FIG. 10). The sequencing run may be a multiplexed run in which multiple different libraries from different sources are pooled together. The different libraries nonetheless share certain common adapter sequences that bind to the sequencing primers disclosed herein. The adapters may also include sequences that vary between samples, e.g., different indexes, that are used to assign a particular sequencing read to a sample or library of origin. The quality metrics may be specific to a particular sample and tied to the index for that sample. In addition, a normalization protocol will allow the user to normalize the entire plate.

The library concentration is calculated per each sample by applying the following formula:

$$Sample 1 [ DNA ] ( nM ) = \% Demux ( sample 1 ) * iSeqQCPool [ DNA ] ( nM )$$

Accordingly, the generated quality control metrics, such as the The same template can be also used to calculate the volumes of sample and resuspension buffer (RSB) needed per sample to normalize the plate at a given volume and concentration. A target normalization concentration (nM) and total normalization volume (μl) can be entered via user input. In the following examples, a target concentration of 2.5 nM and a target total volume of 20 μl were entered.

Examples: An example PCR-Free 450 library (NA12878 gDNA) run with the iSeqQC is described. The metrics used to qualify the TSPF450 library are listed and explained in the following table (table 1). The % cluster PF, % Occupancy and % Q30 bases specifications were based on the iSeq specification sheet released by Illumina. The insert size specification was based on the desirable insert size. The rest of the metrics are based on 6 TS PCR-Free 2×151 iSeqQC runs performed previously with good quality libraries (all tested in Novaseq6000 against the specs).

TABLE 1
Quality control specification values: All the specification
values were based on 6 TSPF450 2 × 151 iSeqQC runs. The
libraries used in these runs were good quality libraries (confirmed
by Novaseq). The specifications were calculated by using the
following formula: Spec = Average ± 3 * Std Dev (±3σ).

| | Specification type | |
|---|---|---|
| | (LSL = lower | |
| | specification limit; | |
| | HSL = higher | Specification |
| iSeq QC metric name | specification limit) | value |
| % clusters PF | LSL | ≥65% |
| (passing filter) | | |
| % Occupancy Mean | LSL | ≥75% |
| (percent wells with at | | |
| least one cluster) | | |
| % Duplicates | HSL | <10% |
| (high adapter dimer | | |
| concentration is | | |
| associated with a | | |
| high duplicate | | |
| percentage) | | |
| % Adapter Dimers | HSL | <2% |
| Insert size** | Range | 400-500 (Insert |
| | | sample ±50) |
| Percent aligned bases | LSL | ≥93 |
| (aligned to human DNA) | | |
| Perc chimeric pairs | HSL | ≤1 |
| Percent read pairs | HSL | ≤1 |
| aligned to different | | |
| chromosomes | | |
| Percent gc content r1 | Range | 40-42 |
| | | (human genome) |
| Percent gc content r2 | Range | 40-42 |
| | | (human genome) |
| Mismatch rate | HSL | ≤2% |
| Percent q30 bases | LSL | ≥80% |
| (base calling accuracy) | | |

LSL: Lower specification limit;
HSL: higher specification limit

Below are the results of quality control example analysis of 5 different samples. Sample 1, 2, 3 and 4 passed all HSL and LSL. Sample 5 failed % PF, % Occupancy, % Duplicates, % Adapter Dimers, % aligned bases and % GC content (for read 1 and 2). This sample QC failure is due to 1% adapter dimers spiked into the pool, therefore, it was expected to fail.

TABLE 2
Quality control results based on specification.

| | Sample | Sample | Sample | Sample | Sample | QC |
|---|---|---|---|---|---|---|
| QC Metrics | 1 | 2 | 3 | 4 | 5* | Specs |
| % PF | 72.19 | 72.19 | 72.19 | 72.19 | 49.81 | >65% |
| % Occupancy | 91.72 | 91.72 | 91.72 | 91.72 | 60.87 | >75% |
| % Duplicates | 2.05 | 2 | 2.03 | 1.81 | 15.23 | <10% |
| % Adapter | 0.6 | 0.7 | 0.22 | 0.38 | 11.80 | <2% |
| dimers | | | | | | |
| Insert size | 432 | 441 | 426 | 447 | 443 | 400-500 |
| Percent | 96.87 | 97 | 97.34 | 97.1 | 89.52 | ≥93 |
| aligned bases | | | | | | |
| % read pairs | 0.75 | 0.66 | 0.76 | 0.71 | 0.88 | <1% |
| aligned to | | | | | | |
| different | | | | | | |
| chromosomes | | | | | | |
| % chimeric | 0.75 | 0.66 | 0.76 | 0.71 | 0.88 | <1% |
| pairs | | | | | | |
| % Q30 bases | 90.18 | 90.46 | 90.59 | 90.16 | 87.40 | >80% |
| % GC Content | 40.78 | 40.7 | 40.62 | 40.62 | 44.53 | [40-42] |
| read1 | | | | | | |
| % GC Content | 40.27 | 40.16 | 40.3 | 40.25 | 44.12 | [40-42] |
| read2 | | | | | | |
| Human | 0.63 | 0.61 | 0.61 | 0.64 | 0.86 | <2% |
| mismatch rate | | | | | | |
| Pass/Fail QC | Y | Y | Y | Y | N | — |

As demonstrated, analysis of sequencing reads from the spiked sample was above the specification for GC content because of the sequencing reads reflected a higher than desired number of sequencing reads generated from adapter dimers. Adapter dimers are synthetic DNA with GC content outside of typical values for human-derived DNA. Therefore, a sequencing library analyzed according to the disclosed techniques with sequencing data indicative of higher-than-desired GC content may be characteristic of the high adapter dimer presence. Together with the other quality metrics that are indicative of high adapter dimer presence, the library can be identified as failing quality control. As also demonstrated, certain metrics, such as insert size, are not flagged or outside of specification limits even in libraries with high adapter dimer presence.

Provided herein are sequencing workflows that detect, e.g., sequence, adapter dimers, and provide this information as input to a quality control analysis. To demonstrate the efficiency of this workflow detecting adapter dimers, a PF450 library was run with different % adapter dimer spiked in. An experiment summary is shown in the following table (Table 3).

TABLE 3
Adapter dimer experiment summary.

| #Z,26 [symbol] | Number of | % Adapter Dimers |
|---|---|---|
| #Z,28 | repeats (n)* | (secondary metrics) |
| 0% (control) | 2 | 0.5 |
| 0.1% | 2 | 12.2 |
| 2% | 2 | 12 |
| 5% | 2 | 27.6 |
| 10% | 2 | 68.6 |

The results confirm that iSeqQC workflow can detect adapter dimers and this detection is sensitive at very low concentrations.

If libraries are combined in unequal concentrations at the pooling step, it can result in biased representation of certain libraries over others. Underrepresentation can require additional sequencing, while overrepresentation can lead to wasted sequencing capacity. Libraries with high amounts of adapter dimers can appear to have sufficient concentration of DNA. However, this concentration may be measuring the presence of the adapter dimers rather than fragments containing and, therefore, may overstate the DNA concentration of DNA from the sample. Assessment of adapter dimer sequencing results can be used to identify a subset of libraries in a multiplexed reaction with a percentage of adapter dimers that does not pass quality control. Such libraries may be provided to a cleanup step and/or may be rebalanced, and may be identified as part of the disclosed techniques. The cleanup step may include a gel or size separation to separate out the adapter dimers from the library. However, because cleanup steps are time consuming, running libraries through quality metrics in conjunction with acquiring sequencing data may permit some libraries to avoid going through cleanup unnecessarily solely on the basis of pre-sequencing analysis, e.g., fragment size data.

Another aspect of the disclosed techniques is that the generated metrics improve rebalancing libraries with a coefficient of variation for the number of counts across all indexes (CV) <10%. Equal index representation can prevent samples failing during sequencing due to low yield. Because the adapter dimers nonetheless include an index sequence that can be represented, e.g., in a first or second index read, library balancing per index sequence will not be accurate for samples with high adapter dimer concentration. Thus, based on index reads directly from adapter dimers, sample representation will be artificially high or overrepresented in a pool based solely on the indexes because some of the % demux comes from the adapter dimer and not the library itself. An improperly balanced sample may then sequence with poor coverage.

This is the most common failure type for high throughput workflow and causes delays in turnaround time and adds sequencing costs. The samples that fail due to low yield will need to be re-sequenced and, in some cases, the library preparation need to be re-made, causing more delays and adding library preparation costs. The iSeq QC workflow allows to control the index representation saving future sequencing time and costs. Using % demux values library can be re-balanced on the plates.

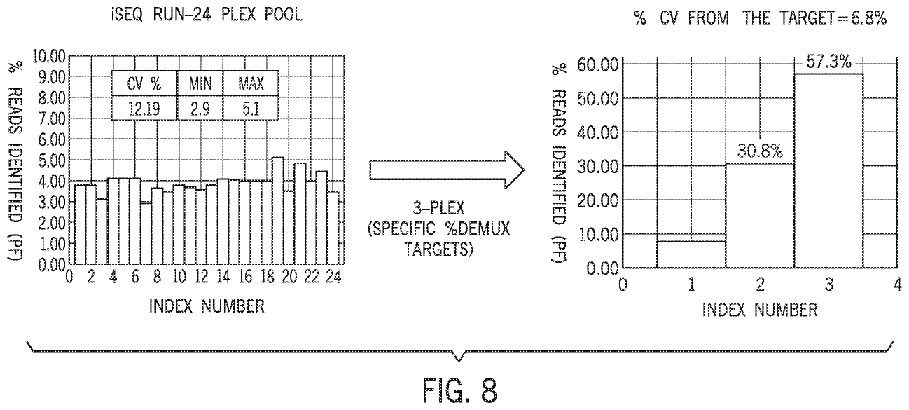

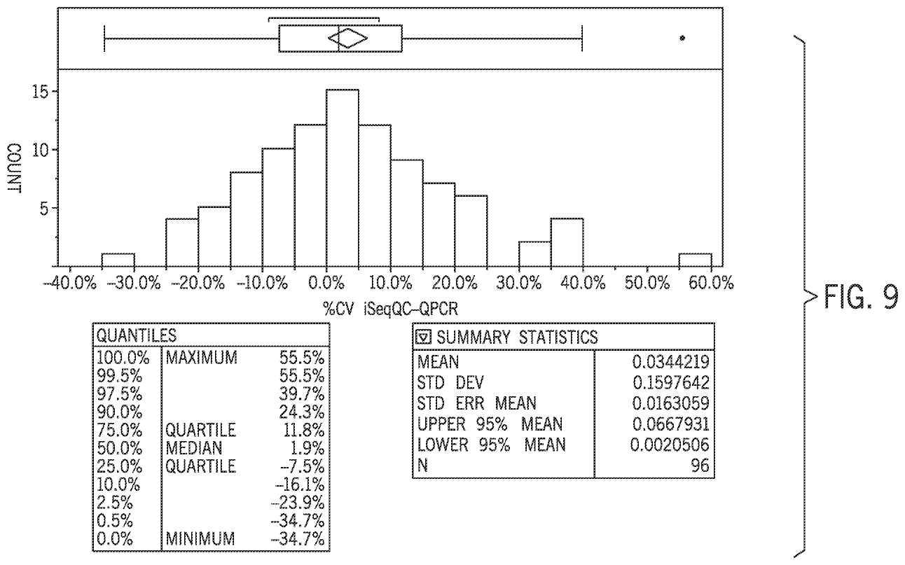

In the next figure, there are examples of libraries rebalanced/normalized based on calculated % demux values. The % CV is very low (<10%) meaning that the % demux values are highly related to DNA concentration and that can be used to re-balance and normalize libraries. As shown in FIG. 8, 24 samples were rebalanced and pooled to produce 2 different library pools with different complexity: 6 plex (A1) and 24 plex (A2). The % CV values for both pools were 7.52% and 9.5% respectively. As shown in FIG. 9, the 24-plex library preparation was used to create a 3-plex pool with different % demux samples per each sample. Library 1 and 2 had 0% CV from the % demux sample (% reads sample). Library 3 had 6.8% CV from the expected % demux sample (% Reads sample). Using the same concept, the concentration for each one of the samples can be calculated as provided herein. These concentration values can be used to normalize the whole plate to a sample concentration and volume.

A comparison between the concentration values generated from the iSeqQC and the concentration from Q-PCR (Roche LightCycler 480, kit KK4953) was performed. FIG. 9 shows the distribution of the % CV between iSeq DNA concentration predictive values and Q-PCR DNA concentration. The % CV average is 3.4%, showing that these is a high correlation between detected Q-PCR DNA concentration and iSeq DNA concentration values. These results show that the DNA concentration calculated using iSeq QC % demux have a high correlation with the Q-PCR DNA concentration values.

The disclosed implementation of a quality control library step permits discarding or modifying of any poor performing library to prevent expending time and money on sequencing this library in larger and relatively expensive sequencing platforms The poor performing library can be subjected to a cleanup step that removes adapter dimers. However, libraries that perform well need not be subjected to such a step, thus saving time for libraries that pass the quality control metrics.

In some embodiments, the disclosed techniques are used to generate a nucleic acid sequencing library (e.g., a library 20) or a DNA fragment library. The generated library can be used in sequencing reactions as provided herein. FIG. 10 is a schematic diagram of a sequencing device 160 that may be used in conjunction with the disclosed embodiments for acquiring sequencing data from indexed nucleic acids (e.g., sequencing reads, read 1, read 2, index reads, index read 1, index read 2, multi-sample sequencing data) that assigned to individual samples using the indexing techniques as provided herein. The sequence device 160 may be implemented according to any sequencing technique, such as those incorporating sequencing-by-synthesis methods described in U.S. Patent Publication Nos. 2007/0166705; 2006/0188901; 2006/0240439; 2006/0281109; 2005/0100900; U.S. Pat. No. 7,057,026; WO 05/065814; WO 06/064199; WO 07/010,251, the disclosures of which are incorporated herein by reference in their entireties. Alternatively, sequencing by ligation techniques may be used in the sequencing device 160. Such techniques use DNA ligase to incorporate oligonucleotides and identify the incorporation of such oligonucleotides and are described in U.S. Pat. Nos. 6,969,488; 6,172,218; and 6,306,597; the disclosures of which are incorporated herein by reference in their entireties. Some embodiments can utilize nanopore sequencing, whereby sample nucleic acid strands, or nucleotides exonucleolytically removed from sample nucleic acids, pass through a nanopore. As the sample nucleic acids or nucleotides pass through the nanopore, each type of base can be identified by measuring fluctuations in the electrical conductance of the pore (U.S. Pat. No. 7,001,792; Soni & Meller, Clin. Chem. 53, 1996-2001 (2007); Healy, Nanomed. 2, 459-481 (2007); and Cockroft, et al. J. Am. Chem. Soc. 130, 818-820 (2008), the disclosures of which are incorporated herein by reference in their entireties). Yet other embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference in its entirety. Particular embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides, or with zeromode waveguides as described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference in their entireties. Other suitable alternative techniques include, for example, fluorescent in situ sequencing (FISSEQ), and Massively Parallel Signature Sequencing (MPSS). In particular embodiments, the sequencing device 160 may be an iSeq from Illumina (La Jolla, CA). In other embodiment, the sequencing device 160 may be configured to operate using a CMOS sensor with nanowells fabricated over photodiodes such that DNA deposition is aligned one-to-one with each photodiode.

The sequencing device 160 may be a “one-channel” detection device, in which only two of four nucleotides are labeled and detectable for any given image. For example, thymine may have a permanent fluorescent label, while adenine uses the same fluorescent label in a detachable form. Guanine may be permanently dark, and cytosine may be initially dark but capable of having a label added during the cycle. Accordingly, each cycle may involve an initial image and a second image in which dye is cleaved from any adenines and added to any cytosines such that only thymine and adenine are detectable in the initial image but only thymine and cytosine are detectable in the second image. Any base that is dark through both images in guanine and any base that is detectable through both images is thymine. A base that is detectable in the first image but not the second is adenine, and a base that is not detectable in the first image but detectable in the second image is cytosine. By combining the information from the initial image and the second image, all four bases are able to be discriminated using one channel. In other embodiments, the sequencing device 160 may be a “two-channel” detection device

In the depicted embodiment, the sequencing device 160 includes a separate sample substrate 162, e.g., a flow cell or sequencing cartridge, and an associated computer 164. However, as noted, these may be implemented as a single device. In the depicted embodiment, the biological sample may be loaded into substrate 162 that is imaged to generate sequence data. For example, reagents that interact with the biological sample fluoresce at particular wavelengths in response to an excitation beam generated by an imaging module 172 and thereby return radiation for imaging. For instance, the fluorescent components may be generated by fluorescently tagged nucleic acids that hybridize to complementary molecules of the components or to fluorescently tagged nucleotides that are incorporated into an oligonucleotide using a polymerase. As will be appreciated by those skilled in the art, the wavelength at which the dyes of the sample are excited and the wavelength at which they fluoresce will depend upon the absorption and emission spectra of the specific dyes. Such returned radiation may propagate back through the directing optics. This retrobeam may generally be directed toward detection optics of the imaging module 172, which may be a camera or other optical detector.

The imaging module detection optics may be based upon any suitable technology, and may be, for example, a charged coupled device (CCD) sensor that generates pixilated image data based upon photons impacting locations in the device. However, it will be understood that any of a variety of other detectors may also be used including, but not limited to, a detector array configured for time delay integration (TDI) operation, a complementary metal oxide semiconductor (CMOS) detector, an avalanche photodiode (APD) detector, a Geiger-mode photon counter, or any other suitable detector. TDI mode detection can be coupled with line scanning as described in U.S. Pat. No. 7,329,860, which is incorporated herein by reference. Other useful detectors are described, for example, in the references provided previously herein in the context of various nucleic acid sequencing methodologies.

The imaging module 172 may be under processor control, e.g., via a processor 174, and may also include I/O controls 176, an internal bus 78, non-volatile memory 180, RAM 82 and any other memory structure such that the memory is capable of storing executable instructions, and other suitable hardware components that may be similar to those described with regard to FIG. 10. Further, the associated computer 164 may also include a processor 184, I/O controls 186, a communications module 84, and a memory architecture including RAM 188 and non-volatile memory 190, such that the memory architecture is capable of storing executable instructions 192. The hardware components may be linked by an internal bus 194, which may also link to the display 196. In embodiments in which the sequencing device 160 is implemented as an all-in-one device, certain redundant hardware elements may be eliminated.

The processor 184 may be programmed to assign individual sequencing reads to a sample based on the associated index sequence or sequences according to the techniques provided herein. In particular embodiments, based on the image data acquired by the imaging module 172, the sequencing device 160 may be configured to generate sequencing data that includes sequence reads for individual clusters, with each sequence read being associated with a particular location on the substrate 170. Each sequence read may be from a fragment containing an insert or may be from an adapter dimer present in the sequencing library. The sequencing data includes base calls for each base of a sequencing read. Further, based on the image data, even for sequencing reads that are performed in series, the individual reads may be linked to the same location via the image data and, therefore, to the same template strand. In this manner, index sequencing reads may be associated with a sequencing read of an insert sequence before being assigned to a sample of origin. The processor 184 may also be programmed to perform downstream analysis on the sequences corresponding to the inserts for a particular sample subsequent to assignment of sequencing reads to the sample.

Further, the sequencing device 160 may generate quality metrics as provided herein and generate reports, notification, and/or data related to the disclosed quality metrics.

The disclosed techniques may be used to sequence a nucleic acid library prepared from a sample nucleic acid (e.g., sample nucleic acid 12). “Sample nucleic acid” can be derived from any in vivo or in vitro source, including from one or multiple cells, tissues, organs, or organisms, whether living or dead, or from any biological or environmental source (e.g., water, air, soil). For example, in some embodiments, the sample nucleic acid comprises or consists of eukaryotic and/or prokaryotic dsDNA that originates or that is derived from humans, animals, plants, fungi, (e.g., molds or yeasts), bacteria, viruses, viroids, mycoplasma, or other microorganisms. In some embodiments, the sample nucleic acid comprises or consists of genomic DNA, subgenomic DNA, chromosomal DNA (e.g., from an isolated chromosome or a portion of a chromosome, e.g., from one or more genes or loci from a chromosome), mitochondrial DNA, chloroplast DNA, plasmid or other episomal-derived DNA (or recombinant DNA contained therein), or double-stranded cDNA made by reverse transcription of RNA using an RNA-dependent DNA polymerase or reverse transcriptase to generate first-strand cDNA and then extending a primer annealed to the first-strand cDNA to generate dsDNA. In some embodiments, the sample nucleic acid comprises multiple dsDNA molecules in or prepared from nucleic acid molecules (e.g., multiple dsDNA molecules in or prepared from genomic DNA or cDNA prepared from RNA in or from a biological (e.g., cell, tissue, organ, organism) or environmental (e.g., water, air, soil, saliva, sputum, urine, feces) source. In some embodiments, the sample nucleic acid is from an in vitro source. For example, in some embodiments, the sample nucleic acid comprises or consists of dsDNA that is prepared in vitro from single-stranded DNA (ssDNA) or from single-stranded or double-stranded RNA (e.g., using methods that are well-known in the art, such as primer extension using a suitable DNA-dependent and/or RNA-dependent DNA polymerase (reverse transcriptase). In some embodiments, the sample nucleic acid comprises or consists of dsDNA that is prepared from all or a portion of one or more double-stranded or single-stranded DNA or RNA molecules using any methods known in the art, including methods for: DNA or RNA amplification (e.g., PCR or reverse-transcriptase-PCR (RT-PCR), transcription-mediated amplification methods, with amplification of all or a portion of one or more nucleic acid molecules); molecular cloning of all or a portion of one or more nucleic acid molecules in a plasmid, fosmid, BAC or other vector that subsequently is replicated in a suitable host cell; or capture of one or more nucleic acid molecules by hybridization, such as by hybridization to DNA probes on an array or microarray.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of characterizing a nucleic acid library comprising:
sequencing a nucleic acid library using a sequencing primer to generate sample sequencing data representative of fragments of the nucleic acid library and of adapter dimer sequencing data, wherein an individual fragment of the nucleic acid library comprises a sample insert flanked by first adapters; wherein an individual adapter dimer of the nucleic acid library comprises second adapters ligated directly to each other at a junction, wherein the first adapters and the second adapters have a same sequence, wherein the sequencing primer is identical to a portion of the same sequence and wherein the individual adapter dimer comprises a mismatch region at the junction and wherein the sequencing primer, when bound to a strand of the individual adapter dimer, has a 3′ terminus that is 5′ of the junction; and
determining a quality metric of the nucleic acid library based on the adapter dimer sequencing data.

2. The method of claim 1, wherein sequencing the nucleic acid library comprises using a mismatch-intolerant polymerase.

3. The method of claim 2, wherein the mismatch-intolerant polymerase is a polymerase have the sequence of SEQ ID NO: 1.

4. The method of claim 2, wherein the mismatch-intolerant polymerase is pol812.

5. The method of claim 1, comprising receiving an input that the nucleic acid library is sequenced to generate the quality metric; and selecting an operating mode of a sequence device that generates the quality metric.

6. The method of claim 1, wherein the sequencing primer has a sequence of SEQ ID NO:2.

7. The method of claim 6, wherein the sequencing primer does not have any nucleotides 3′ of SEQ ID NO:2.

8. The method of claim 1, wherein the sequencing primer has a sequence of SEQ ID NO:3.

9. The method of claim 8, wherein the sequencing primer does not have any nucleotides 3′ of SEQ ID NO:3.

10. The method of claim 1, wherein sequencing the nucleic acid library comprises using an additional sequencing primer, wherein the sequencing primer is used to sequence a first strand of the individual fragment and wherein the additional sequencing primer is used to sequence a reverse strand of of the individual fragment.

11. The method of claim 1, wherein sequencing the nucleic acid library comprises using an additional sequencing primer, wherein the additional sequencing primer is identical to a different portion of the same sequence.

12. The method of claim 1, wherein the sequencing primer is complementary to a location on the first adapters that is separated from the sample insert by at least one nucleotide.

13. The method of claim 12, wherein the sequencing primer is complementary to a location on the first adapters that is separated from the sample insert by one to three nucleotides.

14. A method of characterizing a nucleic acid library comprising:
receiving, at a sequencing device, an input that a sequencing run of a pool of a plurality of nucleic acid libraries is an adapter dimer quality control sequencing run;
causing the sequencing device to generate sequence data from the pool using a sequencing primer that is complementary to a common adapter sequence in fragments of the plurality of nucleic acid libraries and that excludes a 3′ terminal nucleotide of the common adapter sequence at a junction with a fragment insert;
calculating quality metrics for each individual nucleic acid library, wherein the quality metrics comprise a percentage of adapter dimers in each individual nucleic acid library; and
identifying a subset of nucleic acid libraries of the plurality of nucleic acid libraries with a percentage of adapter dimers above a specification limit.

15. The method of claim 14, wherein the sequencing primer terminates within three nucleotides 5′ of the fragment insert in the fragments of the plurality of nucleic acid libraries.

16. The method of claim 14, wherein the sequencing run is a paired end sequencing run, and wherein the sequence data is generated using an additional sequencing primer.

17. The method of claim 14, wherein the 3′ terminal nucleotide of the common adapter sequence is a T.

18. The method of claim 14, wherein the quality metrics further comprise a percentage of duplicate reads, wherein a percent duplicate reads specification high limit is 10%.

19. The method of claim 14, comprising rebalancing nucleic acid libraries in the identified subset.

20. The method of claim 14, comprising estimating a DNA concentration of each nucleic acid libraries of the plurality of nucleic acid libraries based on the quality metrics, wherein the quality metrics further comprise a % coefficient of variation.

21. A sequencing device, comprising:
a flow cell having loaded thereon a pool of a plurality of nucleic acid libraries and a sequencing primer that is complementary to a common adapter sequence in fragments of the plurality of nucleic acid libraries and that excludes a 3′ terminal nucleotide of the common adapter sequence at a junction with a fragment insert;
a computer programmed to:
receive an input that a sequencing run of the pool is an adapter dimer quality control sequencing run;
cause the sequencing device to generate sequence data from the pool using the sequencing primer;
calculate quality metrics for each individual nucleic acid library to determine a percentage of adapter dimers in each individual nucleic acid library; and
identify a subset of nucleic acid libraries of the plurality of nucleic acid libraries with a percentage of adapter dimers above a specification limit.

22. The sequencing device of claim 21, comprising a display that displays the identified subset and the quality metrics.

23. The sequencing device of claim 21, wherein the computer is programmed to generate a notification related to the identified subset.


SEQUENCE LISTING (1 sequence)

SEQ ID NO: 1 - protein, 775 aa, Thermococcus sp.
  SITE (1)..(775): Thermococcus sp. 9 N-7 (Polymerase 812, pol812)
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK